(19) United States
(12) Patent Application Publication
(10) Pub. No.: US 2005/0113804 A1
(43) Pub. Date: May 26, 2005

(54) Variable diameter delivery catheter

(76) Inventors: Cathleen von Lehe, Maple Grove, MN (US); Brooke Ren, Maple Grove, MN (US); Thomas L. Clubb, Hudson, WI (US); Richard S. Kusleika, Eden Prairie, MN (US)

Correspondence Address: POPOVICH, WILES & O'CONNELL, PA;650 THIRD AVENUE SOUTH, SUITE 600, MINNEAPOLIS, MN, 55402, US

(21) Appl. No.: 10/917,249

(22) Filed: Aug. 12, 2004

Related U.S. Application Data
(60) Provisional application No. 60/508,437, filed on Oct. 3, 2003.

(51) Int. Cl. A61M 25/01

(52) U.S. Cl. 604/528

(57) ABSTRACT

The invention provides a catheter that provides storage for an embolic protection device in an accessible, out-of-the-way location within the advancing catheter.

This application claims the benefit of provisional application Ser. No. 60/508,437, filed Oct. 3, 2003, the contents of which are hereby incorporated herein by reference.

### FIELD OF THE INVENTION

This invention relates to devices used in a blood vessel or other lumen in a patient's body. In particular, the present invention relates to delivery catheters having a variable diameter.

### BACKGROUND OF THE INVENTION

Coronary vessels, partially occluded by plaque, may become totally occluded by thrombus or blood clot causing myocardial infarction, angina, and other conditions. Carotid, renal, peripheral, and other blood vessels can also be restrictive to blood flow and require treatment. A number of medical procedures have been developed to allow for the removal or displacement (dilation) of plaque or thrombus from vessel walls to open a channel to restore blood flow and minimize the risk of myocardial infarction. For example, atherectomy or thrombectomy devices can be used to remove atheroma or thrombus. In cases where infusion of drugs or aspiration of thrombus may be desired, infusion or aspiration catheters can be placed near the treatment site to infuse or aspirate. In cases where the treatment device can be reasonably expected to shed emboli, embolic protection devices can be placed near the treatment site to capture and remove emboli. In other cases, a stent is placed at the treatment site. Both embolic protection devices and stents can be placed in or near the treatment site using delivery catheters.

In percutaneous transluminal coronary angioplasty (PTCA), a guide wire and guide catheter are inserted into the femoral artery of a patient near the groin, advanced through the artery, over the aortic arch, and into a coronary artery. An inflatable balloon is then advanced into the coronary artery, across a stenosis or blockage, and the balloon inflated to dilate the blockage and open a flow channel through the partially blocked vessel region. One or more stents may also be placed across the dilated region or regions to structurally maintain the open vessel. Balloon expandable stents are crimped onto a balloon in the deflated state and delivered to the lesion site. Balloon expansion expands the stent against the lesion and arterial wall. Alternatively, self expanding stents can be restrained in a sheath, delivered to the treatment site, and the sheath removed to allow expansion of the stent.

Embolic protection devices have been developed to prevent the downstream travel of materials such as thrombi, grumous, emboli, and plaque fragments. Devices include occlusive devices and filters. Occlusive devices, for example distal inflatable balloon devices, can totally block fluid flow through the vessel. The material trapped by the inflatable devices can remain in place until removal using a method such as aspiration. However, aspiration cannot remove large particles because they will not fit through the aspiration lumen. Also, aspiration is a weak acting force and will not remove a particle unless the tip of the aspirating catheter is very close to the particle to be removed. During the occlusion, the lack of fluid flow can be deleterious. In coronary applications, the lack of perfusing blood flow can cause angina. In carotids, seizure can result from transient blockage of blood flow. In both coronaries and carotids, it is not possible to predict who will suffer from angina or seizure due to vessel occlusion. If a procedure starts with an occlusive device, it may be necessary to remove it and start over with a filter device.

Occlusive embolic protection devices can also be placed proximal to the treatment site. Debris generated at or near the treatment site will not be transported from the treatment site if a proximal occlusive device substantially stops blood flow through the vessel. The material generated during treatment can remain in place until removal using a method such as aspiration. Generally, proximal occlusive embolic protection devices suffer from many of the same limitations as distal occlusive embolic protection devices.

Other embolic protection devices are filters. Filters can allow perfusing blood flow during the emboli capture process. The filters can advance downstream of a site to be treated and expand to increase the filter area. The filter can capture emboli, such as grumous or atheroma fragments, until the procedure is complete or the filter is occluded. When the filter reaches its capacity, the filter may then be retracted and replaced.

Embolic protection devices can be delivered over wires and within guide catheters. The embolic protection methods are normally practiced ancillary to another medical procedure, for example PTCA with stenting or atherectomy. The embolic protection procedure typically protects downstream regions from emboli resulting from practicing the therapeutic interventional procedure. In the example of PTCA, the treating physician must advance a guide wire over the aorta and into a coronary ostium. Advancing the guide wire through tortuous vessels from a femoral artery approach can be difficult and vary with both the patient and the vessel site to be treated. Guide wires are typically selected by the treating physician, based on facts specific to the patient and therapeutic situation, and also on the training, experiences, and preferences of the physician. In particular, a physician may have become very efficient in using a specific guide wire to identify the left coronary ostium and then advance a balloon catheter over the positioned guide wire. The efficacy of the procedure may depend on the physician being able to use a favored guide wire.

In the example PTCA procedure, a guide catheter extends proximally from the patient's groin area, and may be about 100 centimeters long. A 320 cm guidewire is placed in the guide catheter and extended distal of the guide into a coronary vessel, leaving about a 200 cm long guide wire proximal region extending from the guide catheter. The embolic protection device delivery catheter, nominally about 130 cm in length, can advance over the guide wire and within the guide catheter, until a length of guide wire extends from both the guide catheter and delivery catheter. The guide wire can then be retracted and removed from the patient. In some methods, the embolic protection device then advances through and out of the positioned delivery catheter, to the target site to be protected or filtered. In other methods, delivery is accomplished by disposing the embolic protection filter device within the delivery catheter distal region, and advancing the delivery catheter and embolic protection device together within the guide catheter, optionally over the guide wire, and deploying the filter by retracting the delivery catheter while maintaining the position of the filter, thus forcing the filter distally out of the delivery catheter.

Advancement of the delivery catheter over a single length, nominally 170 cm long guide wire presents a problem. The treating physician can only advance the filter delivery catheter about 40 cm over the guide wire until the delivery catheter advances into the patient and the guide wire is inaccessible within the delivery catheter. The guide wire position should be controlled at all times so as to not be dislodged by the advancing delivery catheter from the hard acquired guide wire position within the patient.

One solution to this problem is to use a guide wire at least double the length of the delivery catheter as described above. A 320 cm long guide wire can extend at least about 150 cm from the patient's groin, having an accessible region exposed at all phases of delivery catheter placement. However, the length of the 320 cm guidewire makes manipulating and rotating the guide wire very difficult for the treating physician. Additional personnel can hold the extra length of the guide wire to prevent the added wire length from falling to the floor, where it would become contaminated. However, not all cardiac catheter laboratories have personnel available to maintain control of the long guide wire. In many labs, the physician is working alone in the sterile field. Advancing a device delivery catheter over a positioned, favored, and short (175 cm) guide wire would be inherently more efficacious than requiring use of an unfamiliar, disfavored, or double length guide wire to position the delivery catheter.

Another alternative catheter design is the monorail or rapid exchange type such as that disclosed in U.S. Pat. No. 4,762,129, issued Aug. 9, 1988, to Bonzel. This catheter design utilizes a conventional inflation lumen plus a relatively short parallel guiding or through lumen located at its distal end and passing through the dilatation balloon. Guide wires used with PTCA balloon catheters are typically 175 cm in length and are much easier to keep within the sterile operating field than 300 to 340 cm guide wires. This design enables the short externally accessible rapid exchange guide wire lumen to be threaded over the proximal end of a pre-positioned guide wire without the need for long guide wires.

Still needed in the art are improved designs for rapid exchange delivery catheters. In particular, it would be desirable to have a catheter that provides storage for an embolic protection device in an accessible, out-of-the-way location within the advancing catheter. In such a catheter, the embolic protection device does not interfere with the guide wire, yet is readily accessible for deployment. Additional desired features for an improved catheter include a small distal profile and a smooth transition between the exterior of the guidewire and the tip of the catheter. Both features help to minimize dislodgment of embolic debris during advancement through a vessel and during crossing of a stenosis.

### SUMMARY OF THE INVENTION

The invention provides a catheter that provides storage for an embolic protection device in an accessible, out-of-the-way location within the advancing catheter. In one embodiment, the catheter comprises an elongate tubular body having a proximal portion, a distal portion, a proximal end, a distal end, a lumen extending between the proximal end and the distal end, and a tube wall disposed about the lumen. A first port is disposed in the distal portion of the tubular body and dimensioned to receive a guide wire therethrough, and the first port is formed through the tube wall. The lumen of the tubular body has a first inner diameter at the first port and a second, reduced inner diameter at a point proximal of the first port.

The invention also provides a method for positioning a catheter within a patient's blood vessel, the method comprising: providing a catheter described herein; providing a guide wire having a proximal end and a distal end; advancing the guide wire to a target site within the patient's blood vessel; and advancing the catheter over the guide wire by inserting the guide wire through the catheter lumen between the distal end and the first port.

It is to be understood that both the foregoing general description and the following detailed description are exemplary and explanatory and are intended to provide further explanation of the invention as claimed.

### BRIEF DESCRIPTION OF THE DRAWINGS

FIG. 1 shows an embolic protection device delivery/recovery catheter with a constriction in the inner diameter of the catheter proximal of a guide wire exit port and proximal of a distal exit port.

FIG. 2A shows an embolic protection device delivery/recovery catheter with a toroid insert sized to fit the inner diameter of the catheter at the location indicated in FIG. 1.

FIG. 2B shows an embolic protection device delivery/recovery catheter with a tubular insert sized to fit the inner diameter of the catheter.

FIG. 2C shows an embolic protection device delivery/recovery catheter with a tubular insert sized to fit the inner diameter of the catheter and a reinforced catheter shaft.

FIG. 2D shows an embolic protection device delivery/recovery catheter with an alternate tubular insert sized to fit the inner diameter of the catheter.

FIG. 2E shows an end view of the tubular insert shown in FIG. 2D.

FIG. 2F shows an alternate tubular insert sized to fit the inner diameter of the catheter.

FIG. 2G shows an embolic protection device delivery/recovery catheter with an alternate constriction in the inner diameter of the catheter proximal of a guide wire exit port and proximal of a distal exit port.

FIG. 2H shows an end view of the alternate constriction in the inner diameter of the catheter shown in FIG. 2G.

FIG. 2I shows an embolic protection device delivery/recovery catheter with an alternate constriction in the inner diameter of the catheter proximal of a guide wire exit port and proximal of a distal exit port.

FIG. 2J shows an end view of the alternate constriction in the inner diameter of the catheter shown in FIG. 2I.

FIG. 2K shows an isometric view of an alternate construction of a toroid insert sized to fit the inner diameter of the catheter shown in FIG. 2A.

FIG. 2L shows an isometric view of an alternate construction of a toroid insert sized to fit the inner diameter of the catheter shown in FIG. 2A.

FIG. 2M shows an isometric view of an alternate tubular insert sized to fit the catheter shown in FIG. 2D.

FIG. 3 shows an embolic protection device delivery/recovery catheter with a funnel-shaped insert having its larger outer diameter sized to fit the inner diameter of the catheter at the location indicated in FIG. 1, with the smaller funnel diameter facing proximally.

FIG. 4 shows an embolic protection device delivery/recovery catheter with a funnel-shaped insert as illustrated in FIG. 3, with the smaller funnel diameter facing distally.

FIG. 5 shows an embolic protection device delivery/recovery catheter with an abrupt change in inner diameter.

FIG. 6 shows an embolic protection device delivery/recovery catheter with a gradual change in inner diameter.

FIG. 7A shows an embolic protection device delivery/recovery catheter with a change in inner diameter and a proximal shaft stiffener.

FIG. 7B shows a section view of the catheter shown in FIG. 7A.

FIG. 7C shows an alternate construction of an embolic protection device delivery/recovery catheter with a change in inner diameter and a proximal shaft stiffener.

FIG. 7D shows a section view of the catheter shown in FIG. 7C.

FIG. 8A shows an embolic protection device delivery/recovery catheter with a toggle stop and a distal reduced diameter region.

FIG. 8B shows a section view of the catheter shown in FIG. 8A.

FIGS. 8C and 8CD show an embolic protection device delivery/recovery catheter with a toggle stop and a distal reduced diameter region with an embolic protection device within the catheter

### DETAILED DESCRIPTION OF THE INVENTION

The terms “distal” and “proximal” as used herein refer to the relative position of the guide wire and catheters in a lumen. The most “proximal” point of the catheter is the end of the catheter extending outside the body closest to the physician. The most “distal” point of the catheter is the end of the catheter placed farthest into a body lumen from the entrance site.

The use of the phrases “distal embolic protection device” or “embolic protection device” herein refers to embolic protection devices that are occlusive and/or filtering. The term “embolic protection device” is meant to include devices used to protect a target site and located either proximal to or distal to the treatment site.

This invention provides catheters with a variable inner diameter of the catheter shaft spaced proximally of a guide wire exit port to provide a location or “holding zone” for a distal embolic protection device, such as an embolic filter device. The catheter retains the device in this location during distal advance to the desired intravascular position. In its retained location, the device avoids interference with the guide wire, yet is readily available for deployment when needed.

This invention applies to any catheter used in conjunction with a guide wire or elongate support member for delivery. The concept is universal. Embolic protection device delivery catheters, balloon catheters, and stent delivery catheters with or without a balloon are typical catheters to which the invention can be applied. The concept can also be applied to percutaneous delivery and recovery catheters for atrial appendage occlusion devices, mitral valve remodeling devices, and the like.

The components of the catheters of the invention are made from biocompatible materials such as metals or polymeric materials. If necessary, these metals or polymeric materials can be treated to impart biocompatibility by various surface treatments, as known in the art. Suitable materials include stainless steel, titanium and its alloys, cobalt-chromium-nickel-molybdenum-iron alloy (commercially available under the trade designation ELGILOY™), carbon fiber and its composites, and polymers such as liquid crystal polymers, polyetheretherketone (PEEK), polyimide, polyester, high density polyethylene, PEBAX®, various nylons, and the like. A shape memory or superelastic material such as nitinol or shape memory polymer is also suitable. The size, thickness, and composition of materials are selected for their ability to perform as desired as well as their biocompatibility. It is to be understood that these design elements are known to one of skill in the art.

The material comprising the catheter is preferably at least partially radiopaque. This material can be made radiopaque by plating, or by using core wires, tracer wires, or fillers that have good X-ray absorption characteristics compared to the human body. Marker bands comprised of generally tubular radiopaque metals may be attached to the catheter.

The tip of the catheter may be a generally softer material so as to help prevent damage to a vessel wall as the tip is advanced through the vasculature. Softer materials such as PEBAX®, nylon, rubbers, urethane, silicone, ethylene vinyl acetate, and the like may be attached to the catheter by adhesives, overmolding, heat bonding, solvent bonding, and other techniques known in the art. The tip may have a geometry designed to assist with advancement of the catheter past intraluminal obstructions, such as any of those constructions contained within US 2002/0111649, Rolled Tip Recovery Catheter, the contents of which are hereby incorporated herein in its entirety.

The catheter is generally referred to as an embolic protection delivery/recovery catheter. However, it is contemplated that the embodiments of the catheters described herein may be used solely for delivery, solely for recovery, or for both delivery and recovery.

The embolic protection device should be constructed of material that will not become permanently distorted to its preloaded configuration. If the device, such as an embolic filter device, is of metal, it can desirably be constructed of steel or of a shape-memory material, such as nitinol.

In one embodiment, the invention provides a catheter for the intravascular deployment of a medical device, the catheter comprising: an elongate tubular body having a proximal portion, a distal portion, a proximal end, a distal end, a lumen extending between the proximal end and the distal end, and a tube wall disposed about the lumen. A first port is disposed in the distal portion of the tubular body and dimensioned to receive a guide wire therethrough, and the first port is formed through the tube wall. The lumen of the tubular body has a first inner diameter at the first port and a second, reduced inner diameter at a point proximal of the first port.

The invention also provides a method for positioning a catheter within a patient's blood vessel, the method comprising: providing a catheter described herein; providing a guide wire having a proximal end and a distal end; advancing the guide wire to a target site within the patient's blood vessel; and advancing the catheter over the guide wire by inserting the guide wire through the catheter lumen between the distal end and the first port.

The various embodiments of the invention will now be described in connection with the drawing figures. It should be understood that for purposes of better describing the invention, the drawings have not been made to scale. Further, some of the figures include enlarged or distorted portions for the purpose of showing features that would not otherwise be apparent.

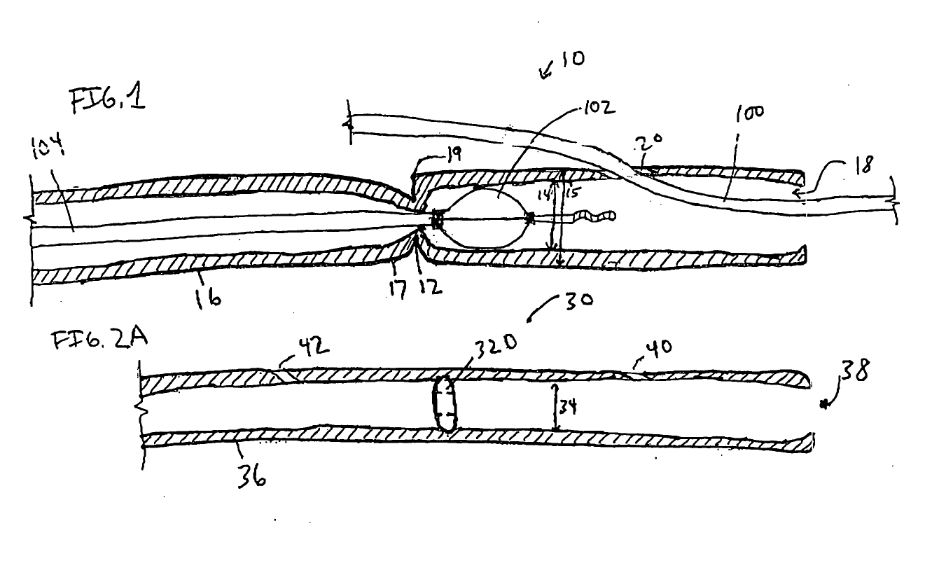

The distal embolic protection device delivery/recovery catheter 10, 30, 50, 70, shown in the FIGS. 1 to 4 embodiments, has a constriction or narrowing 12, 320, 321, 322, 323, 324, 325, 52, 72 in the inner diameter 14, 34, 54, 74 of the catheter shaft 16, 36, 56, 76 proximal of the distal exit port 18, 38, 58, 78 and proximal of the guide wire exit port 20, 40, 60, 80, respectively. The catheter 10, 30, 50, 70 is constructed and designed for use with any suitable guide wire 100. The constriction or narrowing 12, 320, 321, 322, 323, 324, 325, 52, 72 of the catheter inner diameter 14, 34, 54, 74, respectively, creates a preloading stop or “holding zone” location for an embolic protection device, such as an embolic filter 102. This location is distal of the constriction 12, 320, 321,322, 323, 324,325, 52, 72 and proximal of the guide wire exit port 20, 40, 60, 80, respectively, to prevent interaction of the guide wire 100 with the filter 102. The guide wire 100 advances into the distal exit port 18, 38, 58, 78 and out through the guide wire port 20, 40, 60, 80, respectively. The catheter 10, 30, 50, 70 may have the filter 102 or other device positioned or preloaded for out-of-the-way, non-interfering storage before and during distal advancement of the catheter 10, 30, 50, 70 over a primary guide wire 100.

In FIG. 1, the shaft 16 of the catheter 10 has an indentation or reduction 12 of both the inner 14 and outer diameter 15 proximal of both the distal exit port 18 and the guide wire exit port 20. The proximal side of the indentation 12 may be a gradual reduction 17 from the catheter shaft 16 full diameter, while the distal side of the indentation 12 may be an abrupt or right-angled comer 19 reduction. The constriction may also be reversed from the FIG. 1 embodiment, with the indentation 12 distal side having a gradual reduction 17 from the catheter shaft 16 full diameter, while the indentation 12 proximal side has an abrupt or right-angled comer 19 reduction. Alternatively, both sides of the indentation 12 may have a gradual or an abrupt reduction from the full diameter, or the constriction 12 may be formed by any type, shape or method that reduces the diameter of the shaft 16. The catheter 10 may be formed with this indentation 12, for example, by heating and crimping a uniform diameter catheter shaft, for example, with a specially designed tool. Alternatively, a band or wire (not shown) may be slid over the catheter shaft and mechanically deformed by crimping or swaging to effect an indentation, with or without application of heat. This indentation 12 creates a preloading stop or “holding zone” location for an embolic protection device, such as an embolic filter 102. The location is distal of the indentation 12 right-angled corner 19 and proximal of the guide wire exit port 20, and is sized and shaped to accommodate any desired embolic protection device, so that the device does not interfere with the guide wire 100 passing through the guide wire exit port 20. The cross-sectional area of the indentation 12 must be large enough to allow free and easy movement of the filter wire 104, while preventing retraction or passage of the filter 102 proximal of the indentation 12. The catheter 10 can be provided to the physician with the filter 102 or other device preloaded for out-of-the-way, non-interfering storage during distal advancement of the catheter 10.

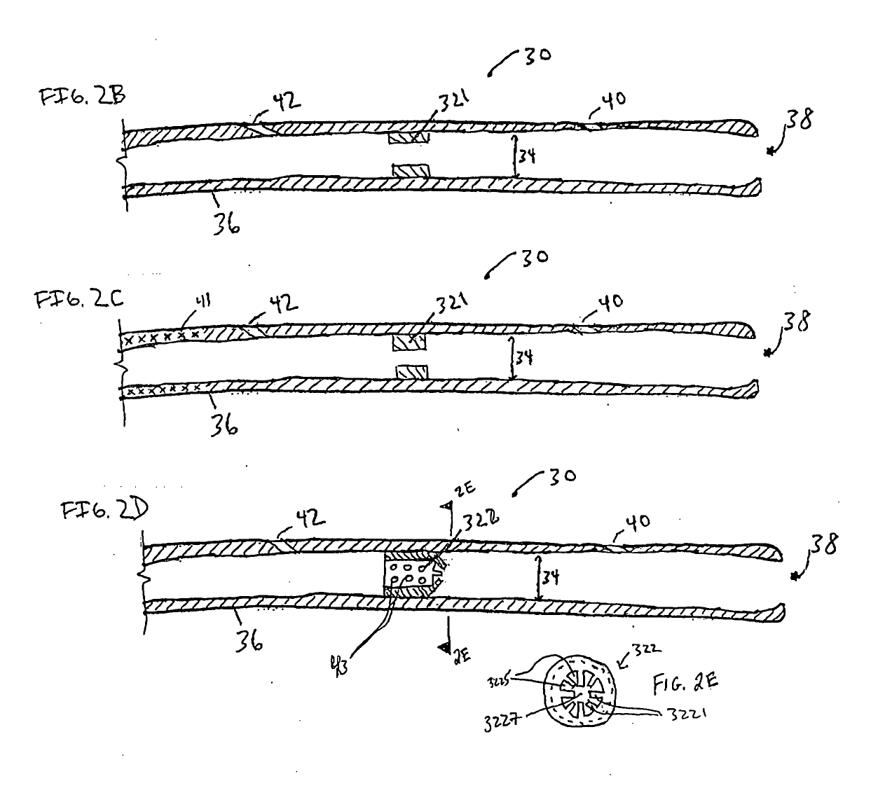

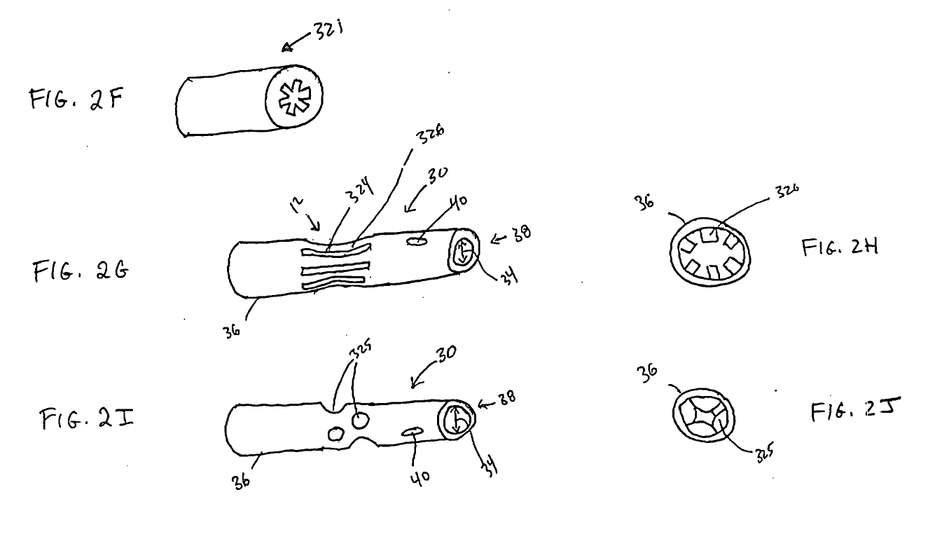

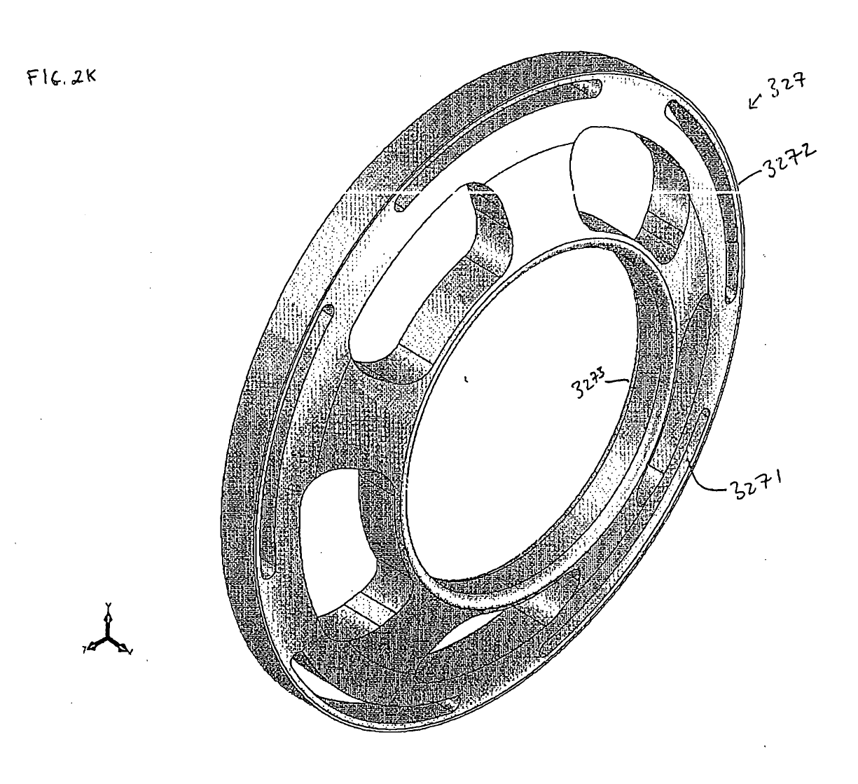

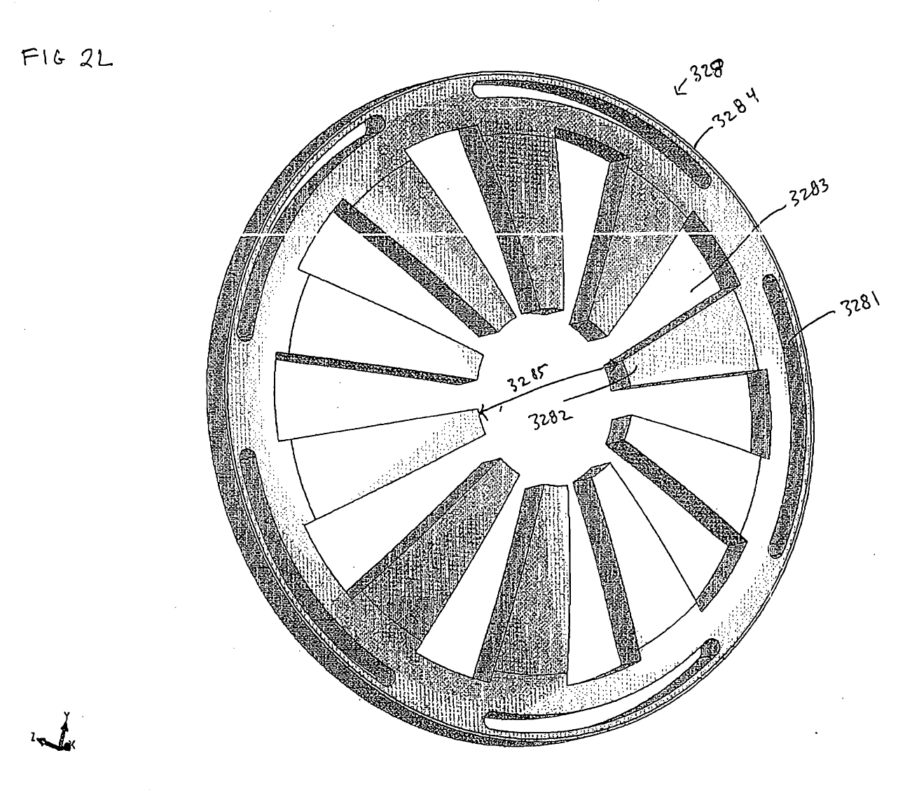

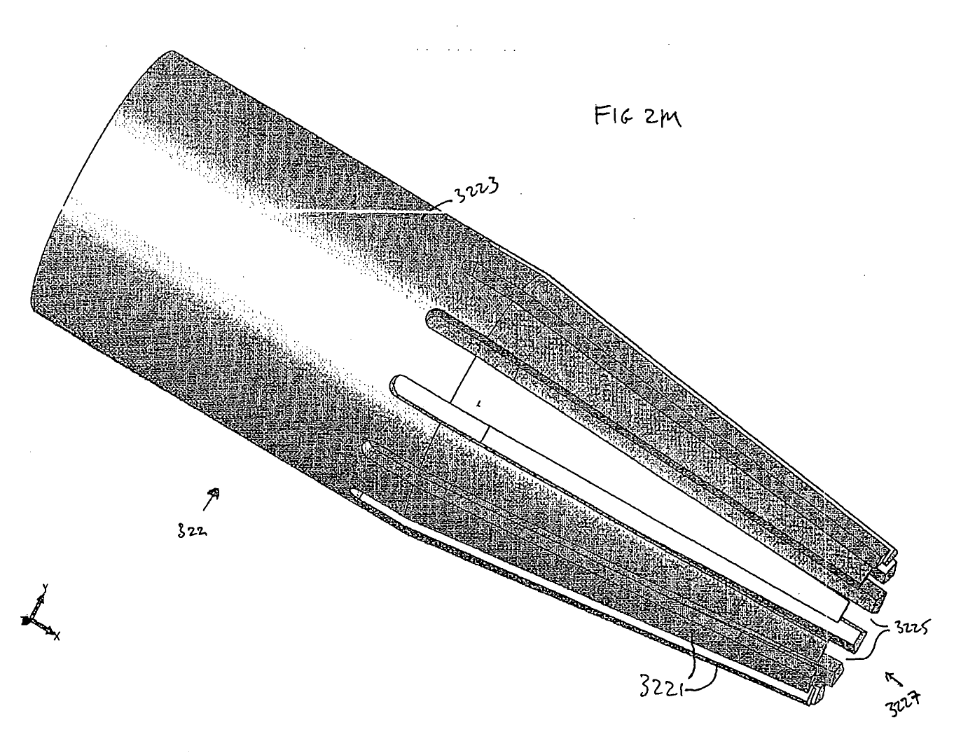

A toroid-shaped insert 320 constricts or narrows the inner diameter 34 of the catheter 30 shaft 36 proximal of the distal exit port 38 and proximal of the guide wire exit port 40 in FIG. 2A. The toroid-shaped insert 32 may be a thin washer, a short length of tubing, or other alternate structures that allow unencumbered passage of filter wire 104 yet prevent passage of filter 102. The walls of the insert 320 may be curvilinear overall (forming a “donut” shape), may form a right cylinder with an axial cylindrical hole, or any other generally toroidal shape. In some embodiments the outer diameter of the insert 320 is sized and shaped to fit tightly to the catheter shaft 36 inner diameter 34. Alternatively, toroid insert 320 may be slightly larger in diameter than catheter shaft 36 inner diameter 34 and anchored within walls of catheter 36. The toroid insert 320 outer diameter may be secured to the inner diameter 34 by any suitable permanent method, such as a press-fit, heat or re-flow bonding, or adhesive bonding. For example, a toroid insert 320 can be inserted into catheter shaft 36 and held at a desired location by mandrels proximal and distal to the insert. The mandrels should be slightly smaller in diameter than catheter inner diameter 34. The catheter region containing insert 320 and the mandrels can be inserted into heat shrink tubing and the assembly heated to shrink the heat shrink tubing, melt the catheter 36 and cause catheter 36 inside diameter 34 to conform to the mandrels, thereby immobilizing insert 320 into the wall of catheter 36. The cross-sectional area of the opening through the toroid 320 must be large enough to allow free and easy passage of the filter wire 104, while preventing proximal retraction of the filter 102 through the toroid 320. The toroid 320 forms a constriction or narrowing 320 of the catheter inner diameter 34 that creates a preloading stop or “holding zone” location for an embolic protection device, such as an embolic filter 102. This location is distal of the toroid 320 and proximal of the guide wire exit port 40. The filter 102 or other device can be preloaded for out-of-the-way or non-interfering storage before and during distal advancement of the catheter 30. In FIG. 2A, a second port 42 can be used as the exit port for the filter wire 104. This second port is optionally incorporated into the catheter 30 and any of the catheter designs disclosed herein can be comprised of this optional second port.

FIG. 2K shows a detailed view of a toroidal insert 327 that constricts or narrows the inner diameter 34 of the catheter 30 shaft 36 proximal of the distal exit port 38 and proximal of the guide wire exit port 40. Toroidal insert 327 may be metal, polymer, ceramic, composite, or any other material that creates a preloading stop or “holding zone” location for an embolic protection device, such as an embolic filter 102. Toroidal insert 327 may have coaxial inside 3273 and outside 3274 diameters (shown), non-coaxial inside 3273 and outside 3274 diameters (not shown), and may have irregular inside or outside diameters. Retaining slot 3271 provides an area for polymer to flow into when fusing the insert to the catheter shaft 36. This flow of polymer into retaining slot 3271 results in an improved bond to the catheter shaft 36.

FIG. 2L shows a toroidal insert 328 that constricts or narrows the inner diameter 34 of the catheter 30 shaft 36 proximal of the distal exit port 38 and proximal of the guide wire exit port 40. Toroidal insert 328 may be metal, polymer, ceramic, composite, or any other material that creates a preloading stop or “holding zone” location for an embolic protection device, such as an embolic filter 102. Toroidal insert 328 may have coaxial inside 3285 and outside 3284 diameters (shown), non-coaxial inside 3285 and outside 3284 diameters (not shown), and may have irregular inside or outside diameters. Retaining slot 3281 provides an area for polymer to flow into when fusing the insert to the catheter shaft 36. This flow of polymer into retaining slot 3281 results in an improved bond to the catheter shaft 36. Toroidal insert 328 is comprised of fingers 3282 and spaces 3283 which cooperate with filter wire 104 and filter 102 to create a preloading stop or “holding zone” location for an embolic protection device, such as an embolic filter 102, as described below in connection with FIGS. 2D, 2E, and 2M. Toroidal insert 328 can be made, for example, by laser cutting a tube or a sheet and is comprised of flexible metals such as stainless steel or nitinol, polymers such as polyester, KEVLAR®, or liquid crystal polymers, ceramics, or other materials capable of elastically deforming without significant deformation in this application.

FIG. 2B shows a tubular insert 321 that constricts or narrows the inner diameter 34 of the catheter 30 shaft 36 proximal of the distal exit port 38 and proximal of the guide wire exit port 40. Tubular insert 321 may be metal, polymer, ceramic, composite, or any other material that creates a preloading stop or “holding zone” location for an embolic protection device, such as an embolic filter 102. Tubular insert 321 may have coaxial inside and outside diameters, non-coaxial inside and outside diameters, and may have irregular inside or outside diameters. One example of a preferred tubular insert 321 with an irregular inside diameter is shown in FIG. 2F. Tubular insert 321 outer diameter may be secured to the inner diameter 34 by any suitable permanent method, such as a press-fit, heat or re-flow bonding, adhesive bonding, or other means as are known in the art.

FIG. 2C shows a catheter 30 similar in many respects to the catheter of FIG. 2B, however the catheter of FIG. 2C is comprised of catheter shaft reinforcement 41. Catheter shaft reinforcement 41 can be comprised of braid, coil, strands, slotted tube, or other shapes that are fused or otherwise bonded into catheter shaft 36 for the purpose of providing bending stiffness and axial stiffness (pushability) to catheter shaft 36. Catheter shaft reinforcement 41 can be comprised of metals such as stainless steel or nitinol, polymers such as polyester, KEVLAR®, or liquid crystal polymers, ceramics, or other materials capable of reinforcing catheter shaft 36.

FIG. 2D shows catheter 30 with tubular fingered insert 322. Tubular fingered insert 322 is anchored to catheter 36 as described in connection with FIG. 2B. Any of the tubular inserts 321, 322 described herein may optionally be provided with holes 43 to assist with anchoring of insert relative to catheter shaft 36. Inside diameter of fingered insert 322 may be as smaller than, equal to, or slightly larger than inside diameter 34 of catheter 36.

Tubular fingered insert 322 is shown in greater detail in FIGS. 2E and 2M. Fingered insert 322 is comprised of at least 2 fingers 3221 attached to a tubular section 3223 and separated by slots 3225. Tubular section 3223 is attached to catheter 36, fingers 3221 are flexible and can radially flex relative to tubular section 3223. The angle of fingers 3221 relative to the central axis of tubular section 3223 can be varied to suit the particular dimensions of catheter 36, filter wire 104, and filter 102 to effect the needed performance. Fingered insert 322 can be made, for example, by laser cutting a tube and is comprised of flexible metals such as stainless steel or nitinol, polymers such as polyester, KEVLAR®, or liquid crystal polymers, ceramics, or other materials capable of elastically deforming without significant deformation in this application. Tubular fingered insert 322 is comprised of end opening 3227 large enough to allow free and easy passage of the filter wire 104, while preventing proximal retraction of the filter 102 through the fingered insert 322. Fingered insert 322 can be configured to create a preloading stop or “holding zone” location for an embolic protection device, such as an embolic filter 102.

In use, filter wire 104 is back loaded through end opening 3227 and filter wire is advanced proximally until filter 102 contacts fingers 3221. Further proximal advancement of filter 102 causes fingers 3221 to deflect towards the central axis of catheter 36 and thereby prevent further proximal advancement of filter 102. From this position, distal advancement of filter 102 allows deflection of fingers 3221 to reverse, allowing distal movement of filter 102 and of filter wire 104 through end opening 3227.

FIGS. 2G and 2H show catheter 30 in which the catheter shaft 36 comprises at least two slots 324 and at least two strips 326. Strips 326 are displaced radially inwardly relative to catheter shaft 36 axis such that catheter shaft inside diameter 34 has a constriction 12 of the inside diameter 34 proximal of both the distal exit port 18 and the guide wire exit port 40. Constriction 12 may be formed by applying heat to deform strips 326 or by other means. Constriction 12 creates a preloading stop or “holding zone” location for an embolic protection device, such as an embolic filter 102. The location is distal of the constriction 12 and proximal of the guide wire exit port 40, and is sized and shaped to accommodate any desired embolic protection device, so that the device does not interfere with the guide wire 100 passing through the guide wire exit port 40. The cross-sectional area of the constriction 12 must be large enough to allow free and easy movement of the filter wire 104, while preventing retraction or passage of the filter 102 proximal of the constriction 12. The catheter 30 can be provided to the physician with the filter 102 or other device preloaded for out-of-the-way, non-interfering storage during distal advancement of the catheter 30.

FIGS. 2I and 2J show catheter 30 in which the catheter shaft 36 comprises at least two indentations 325. Indentations 325 are displaced radially inwardly relative to catheter shaft 36 axis such that catheter shaft inside diameter 34 has a constriction 12 of the inside diameter 34 proximal of both the distal exit port 38 and the guide wire exit port 40. Constriction 12 may be formed by applying heat to deform indentations 325 or by other means. Constriction 12 creates a preloading stop or “holding zone” location for an embolic protection device, such as an embolic filter 102. The location is distal of the constriction 12 and proximal of the guide wire exit port 40, and is sized and shaped to accommodate any desired embolic protection device, so that the device does not interfere with the guide wire 100 passing through the guide wire exit port 40. The cross-sectional area of the constriction 12 must be large enough to allow free and easy movement of the filter wire 104, while preventing retraction or passage of the filter 102 proximal of the constriction 12. The catheter 30 can be provided to the physician with the filter 102 or other device preloaded for out-of-the-way, non-interfering storage during distal advancement of the catheter 30.

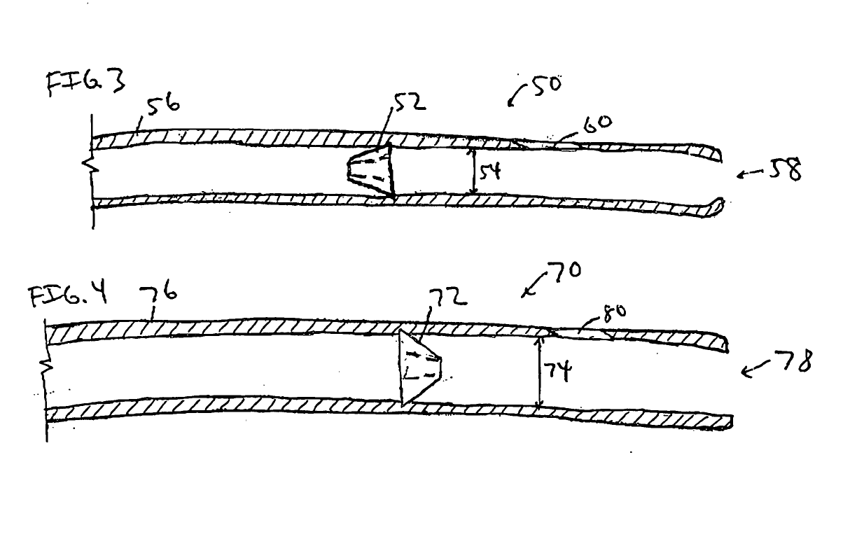

In FIGS. 3 and 4, a funnel-shaped member 52, 72 provides a constriction or narrowing of the inner diameter 54, 74 of the shaft 56, 76 of the catheter 50, 70, respectively. The funnel-shaped member 52, 72 is proximal of both the distal exit port 58, 78 and the guide wire exit port 60, 80, respectively, and may be made of metal, polymer, ceramic, composite, or any other material that creates a preloading stop or “holding zone” location for an embolic protection device, such as an embolic filter 102. The larger outer diameter of the funnel-shaped member 52, 72 is sized and shaped to fit tightly to the catheter shaft 56, 76 inner diameter 54, 74, respectively. The funnel-shaped member 52, 72 outer diameter may be affixed to the inner diameter 54, 74, respectively, by any suitable permanent method, such as a press-fit, heat or re-flow bonding, or adhesive bonding. For example, funnel-shaped member 52, 72 can be inserted into catheter shaft 56, 76 and held at a desired location by mandrels proximal and distal to the member. The mandrels should be slightly smaller in diameter than catheter inner diameter 54, 74. The catheter region containing funnel-shaped member 52, 72 and the mandrels can be inserted into heat shrink tubing and the assembly heated to shrink the heat shrink tubing, melt the catheter shaft 56, 76 and cause catheter shaft 56, 76 inside diameter 54, 74 to conform to the mandrels, thereby immobilizing funnel-shaped member 52, 72 into the wall of catheter shaft 56, 76. The inner opening of the member 52, 72 may be funnel-shaped corresponding to the exterior shape of the member 52, 72. Alternatively, the inner opening may be cylindrical or any other suitable shape. The cross-sectional area of the opening of the funnel-shaped member 52, 72 must allow free and easy passage of the filter wire 104, while preventing proximal retraction of the filter 102 through the member 52, 72.

In the FIG. 3 embodiment 50, the smaller diameter of the funnel-shaped member 52 faces proximally, and in the FIG. 4 embodiment 70, the smaller diameter of the funnel-shaped member 72 faces distally. The funnel-shaped member 52, 72 in the catheter inner diameter 54, 74, respectively, creates a preloading stop or “holding zone” location for a distal embolic protection device, such as an embolic filter 102. The filter 102 or other device can be preloaded to be non-interfering with the guide wire 100 through the guide wire exit port 60, 80.

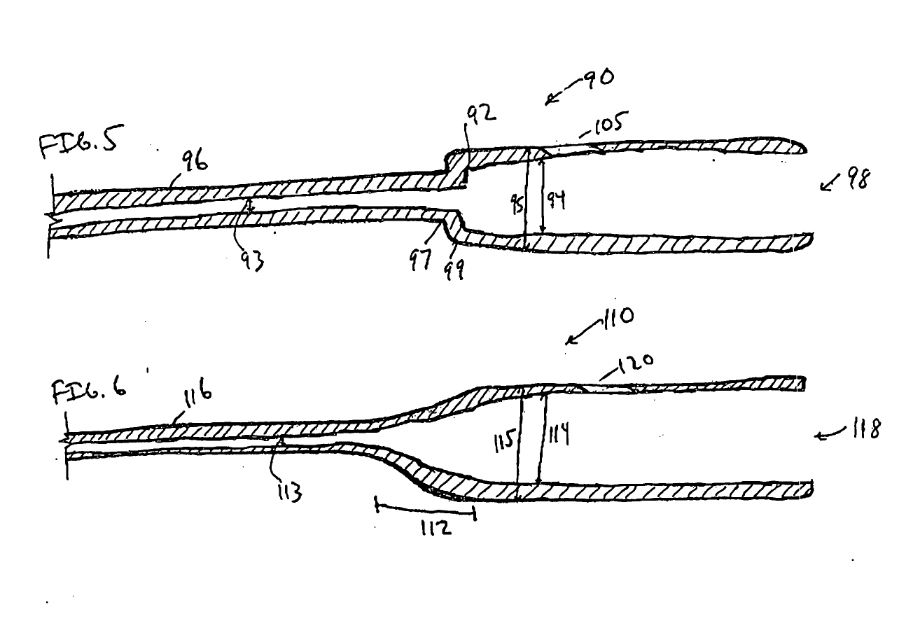

The embolic protection device delivery/recovery catheter 90, 110 shown in the FIGS. 5 and 6 embodiments, has a constriction or narrowing 92, 112 in the inner diameter 94, 114 of the catheter shaft 96, 116 proximal of the distal exit port 98, 118 and proximal of the guide wire exit port 105, 120, respectively. The axes of the inner diameter 94, 114 of the catheter shaft and the inner diameter 93, 113 of the proximal portion of the catheter shaft may be substantially coaxial as shown in FIGS. 5 and 6 or may be offset and parallel (not shown). The catheter 90, 110 is constructed and designed for use with any suitable guide wire 100. The constriction or narrowing 92, 112 of the catheter inner diameter 94, 114, respectively, creates a preloading stop or “holding zone” location for a distal embolic protection device, such as an embolic filter 102. This location is distal of the constriction 92, 112 and proximal of the guide wire exit port 105, 120, respectively, to prevent interaction of the guide wire 100 with the filter 102. The guide wire 100 advances into the distal exit port 98, 118 and out through the guide wire port 105, 120, respectively. The catheter 90, 110 may have the filter 102 or other device positioned or preloaded for out-of-the-way, non-interfering storage before and during distal advancement of the catheter 90, 110 over a primary guide wire 100.

The catheter of FIGS. 5 and 6 has the advantage of providing transverse support to filter wire 104. It is advantageous to taper the diameter of filter wire 104 such that the diameter of the filter wire 104 near the filter 102 is reduced compared to the diameter of the filter wire 5-20 cm proximal to the filter 102. Filter wires so tapered can buckle when they are used to distally advance a filter out of a catheter such catheter 90, 110 respectively. By reducing the inner diameter 93, 113 of the proximal portion of the catheter shaft 96, 116 respectively, lateral support is provided to a tapered filter wire 104 during distal advancement of filter 102 from the catheter. Said lateral support can help prevent filter wire 104 buckling.

In FIGS. 5 and 6, the shaft 96, 116 of the catheter 90, 110 has an indentation or reduction 92, 112 of both the inner 94, 114 and outer diameter 95, 115 proximal of both the distal exit port 98, 118 and the guide wire exit port 105, 120. In FIG. 5, the proximal side of the indentation 92 is an abrupt or right-angled corner 97 reduction from the catheter shaft 96 full diameter, and the distal side of the indentation 92 is an abrupt or right-angled comer 99 reduction. The inner diameter 93 of the proximal portion of the shift 6 is less than the inner diameter 94 of the distal portion of the shaft. In FIG. 6, the reduction 112 is a gradual reduction from the catheter shaft 116's largest inner diameter 114. The inner diameter 113 of the proximal portion of the shaft 116 is less than the inner diameter 114 of the distal portion of the shaft.

The reduction or indentation 92, 112 creates a preloading stop or “holding zone” location for a distal embolic protection device, such as an embolic filter 102. The location is distal of the reduction 92, 112 and proximal of the guide wire exit port 105, 120, and is sized and shaped to accommodate any desired distal embolic protection device or other device, so that the device does not interfere with the guide wire 100 passing through the guide wire exit port 105, 120. The cross-sectional area of the indentation 92, 112 at its narrowest point must be large enough to allow free and easy movement of the filter wire 104, while preventing retraction or passage of the filter 102 proximal of the indentation 92, 112. The catheter 90, 110 can be provided to the physician with the filter 102 or other device preloaded for out-of-the-way, non-interfering storage during distal advancement of the catheter 90, 110.

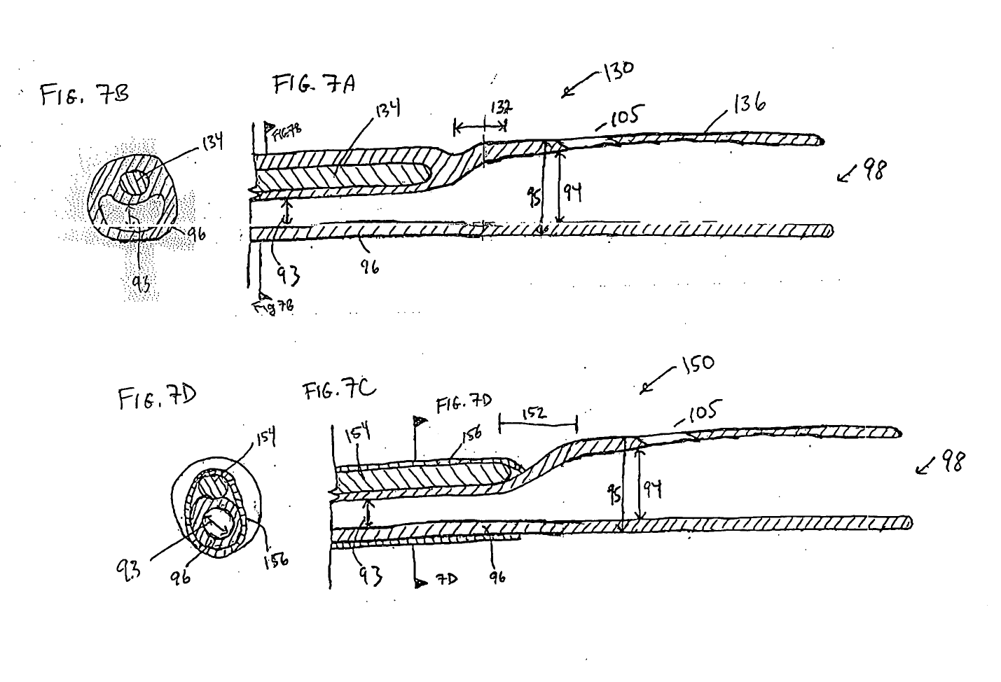

The embolic protection device delivery/recovery catheter 130 shown in FIGS. 7A and 7B has a constriction or narrowing 132 in the inner diameter 94 of the catheter shaft 96 proximal of the distal exit port 98 and proximal of the guide wire exit port 105. The axes of the inner diameter 94 of the catheter shaft and the inner diameter 93 of the proximal portion of the catheter shaft are offset and substantially parallel. The catheter 130 is constructed and designed for use with any suitable guide wire 100. The constriction or narrowing 132 of the catheter inner diameter 94 creates a preloading stop or “holding zone” location for a distal embolic protection device, such as an embolic filter 102. This location is distal of the constriction 132 and proximal of the guide wire exit port 105 to prevent interaction of the guide wire 100 with the filter 102. The guide wire 100 advances into the distal exit port 98 and out through the guide wire port 105. The catheter 130 may have the filter 102 or other device positioned or preloaded for out-of-the-way, non-interfering storage before and during distal advancement of the catheter 130 over a primary guide wire 100.

Additionally, the embolic protection device delivery/recovery catheter 130 shown in FIGS. 7A and 7B, has a stiffening member 134 embedded in wall of catheter 96. Stiffening member 134 may comprise metal, polymer, ceramic, composite, or any other material that imparts bending stiffness and columnar stiffness to proximal portion of catheter shaft 96 for the purpose of improved catheter pushability and trackability through the vasculature of a patient. By way of example, catheter shaft 96 may be comprised of a two lumen extrusion as shown in FIG. 7B with stiffening member 134 within one of the lumens. Distal portion 136 of catheter 96 may be a single lumen tube attached to two lumen tube of catheter 96 by heat fusing by means of mandrels and heat shrink tubing in a manner similar to that described above in connection with FIG. 2A using methods well known to those of skill in the art.

The embolic protection device delivery/recovery catheter 150 shown in FIGS. 7C and 7D has a constriction or narrowing 152 in the inner diameter 94 of the catheter shaft 96 proximal of the distal exit port 98 and proximal of the guide wire exit port 105. The axes of the inner diameter 94 of the catheter shaft and the inner diameter 93 of the proximal portion of the catheter shaft are offset and substantially parallel. The catheter 150 is constructed and designed for use with any suitable guide wire 100. The constriction or narrowing 152 of the catheter inner diameter 94 creates a preloading stop or “holding zone” location for a distal embolic protection device, such as an embolic filter 102. This location is distal of the constriction 152 and proximal of the guide wire exit port 105 to prevent interaction of the guide wire 100 with the filter 102. The guide wire 100 advances into the distal exit port 98 and out through the guide wire port 105. The catheter 150 may have the filter 102 or other device positioned or preloaded for out-of-the-way, non-interfering storage before and during distal advancement of the catheter 150 over a primary guide wire 100.

The catheter of FIGS. 7A to 7D has the advantage of providing transverse support to filter wire 104. It is advantageous to taper the diameter of filter wire 104 such that the diameter of the filter wire 104 near the filter 102 is reduced compared to the diameter of the filter wire 5-20 cm proximal to the filter 102. Filter wires so tapered can buckle when they are used to distally advance a filter out of a catheter such catheter 130, 150 respectively. By reducing the inner diameter 93 of the proximal portion of the catheter shaft 96, lateral support is provided to a tapered filter wire 104 during distal advancement of filter 102 from the catheter. Said lateral support can help prevent filter wire 104 buckling.

Additionally, the embolic protection device delivery/recovery catheter 150 shown in FIGS. 7C and 7D, has a stiffening member 154 embedded in wall of catheter 96. Stiffening member 134 may comprise metal, polymer, ceramic, composite, or any other material that imparts bending stiffness and columnar stiffness to proximal portion of catheter shaft 96 for the purpose of improving catheter pushability and trackability through the vasculature of a patient. By way of example, catheter shaft 96 may be comprised of a heat shrink tubing as shown in FIG. 7D with stiffening member 134 and catheter shaft within the lumen of the heat shrink tubing 156 and held in close apposition thereby.

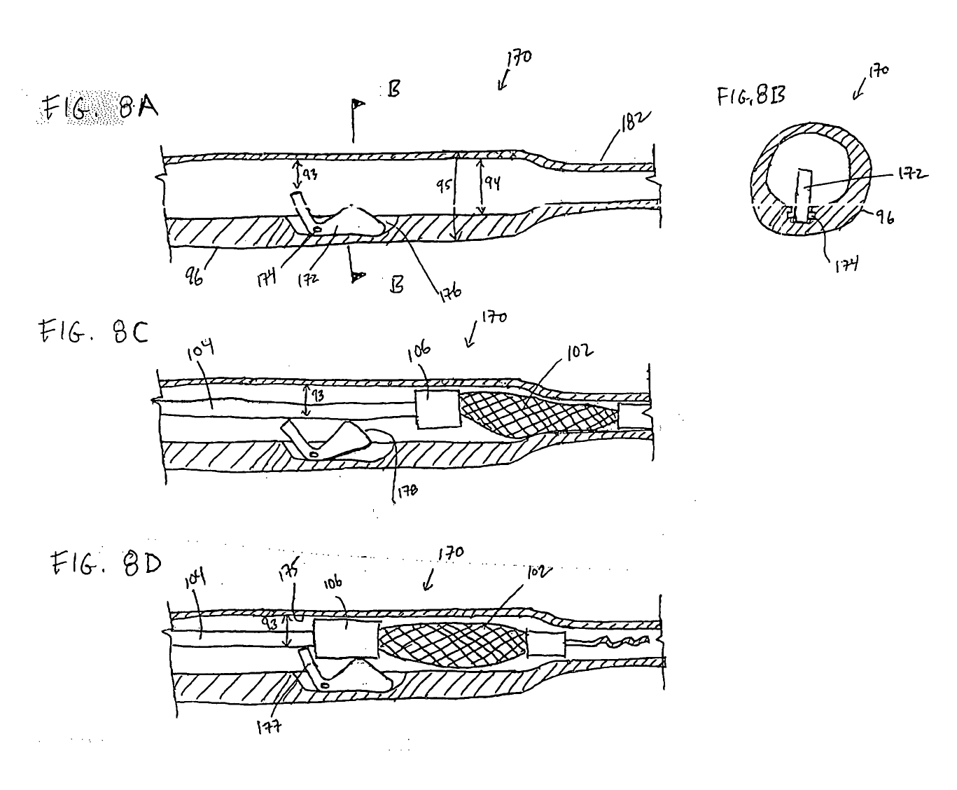

The embolic protection device delivery/recovery catheter 170 shown in FIGS. 8A to 8D has a constriction or narrowing effected by toggle 172 in the inner diameter 94 of the catheter shaft 96 proximal of the distal exit port (not shown) and proximal of the guide wire exit port (not shown). The axes of the inner diameter 94 of the catheter shaft and the effective inner diameter 93 of the proximal portion of the catheter shaft are offset and substantially parallel. The catheter 170 is constructed and designed for use with any suitable guide wire 100. The constriction or narrowing effected by toggle 172 creates a preloading stop or “holding zone” location for a distal embolic protection device, such as an embolic filter 102. This location is distal of the constriction effected by toggle 172 and proximal of the guide wire exit port to prevent interaction of the guide wire 100 with the filter 102. The guide wire 100 advances into the distal exit port and out through the guide wire port. The catheter 170 may have the filter 102 or other device positioned or preloaded for out-of-the-way, non-interfering storage before and during distal advancement of the catheter 170 over a primary guide wire 100. Additionally, the embolic protection device delivery/recovery catheter 170 shown in FIGS. 8A to 8D has a distal diameter reduced portion 182 of catheter 96. Diameter reduced portion 182 of catheter 96 may be formed by necking, swaging, or other means as are known in the art. Diameter reduced portion 182 of catheter 96 advantageously provides a reduced lesion crossing profile to catheter 96. Any of the catheters described herein may be comprised of diameter reduced portion 182.

Toggle 172 and toggle pivot 174 may comprise metal, polymer, ceramic, composite, or any other material that has enough strength to prevent passage of filter proximally past toggle 172. Toggle pivot is embedded in catheter 96 within pocket 176. Pocket 176 allows toggle to move relatively freely about toggle pin 174. Catheter 96 may be reinforced (not shown), for example with metals, in the vicinity of toggle pin to prevent toggle pin 174 from tearing out of catheter 96 during use.

Toggle 172 effects a constriction or narrowing and thereby creates a preloading stop or “holding zone” location for a distal embolic protection device, such as an embolic filter 102 as follows. Filter wire is backloaded into distal exit port (not shown) and past toggle 172 as shown in FIG. 8C. As filter wire 104 traverses toggle 172 toggle will pivot, allowing filter wire 104 to pass through effective inner diameter 93. Further proximal advancement of filter wire 104 will cause enlarged proximal end of filter 106 to contact distal face 178 of toggle, and still further proximal advancement of filter wire 104 will cause causing toggle 172 to pivot about toggle pin 174 and decrease effective inner diameter 93 by moving proximal toggle arm 177 towards the opposing wall 175 of catheter 96. Proximal advancement of filter wire 104 will cease when enlarged proximal end of filter 106 contacts proximal toggle arm 177.

The following general details of the construction and operation of the inventive catheter apply to all embodiments, with specific details for individual wire exit port located from 5 to 30 cm from the catheter distal tip. Proximal of embodiments as noted. Preferably, the catheter of this invention has a guide wire exit port located from 5 to 30 cm from the catheter distal tip. Proximal of the guide wire exit port is a constriction that creates a reduction of the size of the inner diameter of the catheter shaft. The distance between the guide wire exit port and the constriction can be made to accommodate the size and shape of the specific distal embolic protection device or other device to be retained.

The catheter inner diameter can be reduced or necked down by any suitable configuration of the overall cross-sectional area that will permit unimpeded passage for a distal embolic protection device wire, while preventing retraction of the device proximal of the constriction. The constriction or diameter reduction can be abrupt, gradual or tapered, or any combination or multiple series of abrupt or gradual tapers or reductions. Additional non-limiting examples of the desired constriction include indentations or dimples within the catheter wall, an intraluminal net or meshwork, or use of a pin transverse to the catheter axis. Additional guide wire exit port(s) may be located proximal of this constriction or diameter reduction.

An exemplar use of the catheters described herein is as follows. A guide catheter is introduced from the groin of the patient, through the femoral artery, and to the ostium of a coronary vessel as previously described and as is well known in the art. A coronary guidewire is threaded through the guidewire and into a coronary vessel to a region of interest. An embolic protection device filter wire 104 is back loaded into the distal exit port of an inventive catheter, through the constriction or narrowing, and proximally through the inventive catheter. The filter wire is advanced proximally until the filter 102 is positioned or preloaded within the catheter and abuts the distal portion of the constriction or narrowing in a preloaded, out-of-the-way, non-interfering storage position. The coronary guidewire is next back loaded into the distal exit port of an inventive catheter and out of the catheter through the guide wire port located distal to the constriction or narrowing. Next the inventive catheter is advanced distally along the guidewire to a region of interest. The guidewire is withdrawn from the patient and catheter is withdrawn proximally relative to the embolic filter 102, whereby the filter deploys or is deployed and the inventive catheter is withdrawn from the patient.

To recover the embolic device the proximal end of the filter wire 104 is back loaded into the distal exit port of an inventive catheter and the catheter advanced distally to the immediate proximity of the filter. The filter is then drawn into the inventive catheter and the inventive catheter removed from the patient.

The catheter of this invention provides many advantages for the physician and the patient. The catheter inner diameter constriction provides a location to preload an embolic protection device and allows the physician to use a guide wire of choice to position the catheter intravascularly. Typical over-the-wire or rapid-exchange catheter designs may allow a physician to use a favored guide wire for catheter positioning, but do not provide a preloaded device in a non-interfering position, as does the present catheter. The catheter may be constructed to accept any type, shape or size of embolic protection device or other device. The physician may obtain the catheter with a preloaded device of choice. The use of the catheter with a preloaded device reduces the distance the catheter must travel, in comparison to a conventional delivery/recovery catheter, thus reducing intravascular manipulation by reducing the number of catheter exchanges, lessening trauma to the patient, and the length of time for the procedure. The catheter with a preloaded device allows correct positioning of the embolic device every time, while preventing interaction of the guide wire with the device. The present catheter improves overall ease of use both in construction of the catheter, in positioning the catheter within the patient, and in deploying the embolic protection device.

The above description and the drawings are provided for the purpose of describing embodiments of the invention and are not intended to limit the scope of the invention in any way. It will be apparent to those skilled in the art that various modifications and variations can be made without departing from the spirit or scope of the invention. Thus, it is intended that the present invention cover the modifications and variations of this invention provided they come within the scope of the appended claims and their equivalents.

1. A catheter for the intravascular deployment of a medical device, the catheter comprising:
an elongate tubular body having a proximal portion, a distal portion, a proximal end, a distal end, a lumen extending between the proximal end and the distal end, and a tube wall disposed about the lumen;
a first port disposed in the distal portion of the tubular body and dimensioned to receive a guide wire therethrough, the first port being formed through the tube wall;
and the lumen of the tubular body having a first inner diameter at the first port and a second, reduced inner diameter at a point proximal of the first port.

2. A catheter of claim 1, wherein the tube wall of the elongate tubular body having a substantially uniform wall thickness.

3. A catheter of claim 1, wherein the tubular body is crimped at a point proximal of the first port to reduce the inner diameter of the lumen of the tubular body at the crimped point.

4. A catheter of claim 1, wherein a toroid-shaped insert is disposed in the lumen of the tubular body perpendicular to the longitudinal axis of the lumen and proximal of the first port to reduce the inner diameter of the lumen of the tubular body.

5. A catheter of claim 4, wherein the toroid is a washer.

6. A catheter of claim 1, wherein a funnel-shaped insert is disposed in the lumen of the tubular body along the longitudinal axis of the lumen and proximal of the first port to reduce the inner diameter of the lumen of the tubular body.

7. A catheter of claim 6, wherein the funnel-shaped insert has 2 larger opening directed towards the distal end of the elongate tubular body.

8. A catheter of claim 6, wherein the funnel-shaped insert has a larger opening directed towards the proximal end of the elongate tubular body.

9. A catheter of claim 1, wherein the lumen of the tubular body has the second, reduced inner diameter over a substantial portion of its length.

10. A catheter of claim 1, wherein the transition from the first inner diameter to the second inner diameter is abrupt.

11. A catheter of claim 1, wherein the transition from the first inner diameter to the second inner diameter is gradual.

12. A catheter of claim 1, wherein the elongate tubular body has a second port disposed in the distal portion of the tubular body and adapted to receive an elongate support element for the medical device, the second port being formed through the tube wall and being disposed proximal to the transition from the first inner diameter to the second inner diameter.

13. A catheter of claim 1, wherein the first port is disposed from 5 to 30 centimeters from the distal end of the elongate tubular body.

14. A catheter of claim 1, wherein the second port is disposed from 5 to 20 centimeters proximal of the first port.

15. A catheter of claim 1, wherein the first port has a maximum dimension of less than 0.040 inch. 16. A catheter of claim 1, wherein the first port has a maximum dimension of less than 0.020 inch. 17. A catheter of claim 1, wherein the catheter has an outer diameter of less than 0.040 inch. 18. An assembly comprising a guide wire and a catheter of claim 1. 19. An assembly comprising a guide wire, a distal embolic protection device on an elongate support element, and a catheter of claim 1. 20. A method for positioning a catheter within a patient's blood vessel, the method comprising:
providing a catheter of claim 1,
providing a guide wire having a proximal end and a distal end;
advancing the guide wire to a target site within the patient's blood vessel; and
advancing the catheter over the guide wire by inserting the guide wire through the catheter lumen between the distal end and the first port.
